(19) United States
(12) Patent Application Publication
(10) Pub. No.: US 2012/0271043 A1
(43) Pub. Date: Oct. 25, 2012

(54) PROCESS AND DEVICE FOR COLLECTING NUCLEIC ACIDS OF MICROORGANISMS FROM A PARTICULATE SAMPLE

(76) Inventors: John Steichen, Landenberg, PA (US); Daniel Demarco, Wilmington, DE (US); Stephen Varkey, Newark, DE (US)

(21) Appl. No.: 13/387,933

(22) PCT Filed: Aug. 2, 2010
(86) PCT No.: PCT/US10/44083

§ 371 Date: Mar. 30, 2012

Related U.S. Application Data
(60) Provisional application No. 61/231,146, filed on Aug. 4, 2009.

(51) Int. Cl. C07H 21/00

(52) U.S. Cl. 536/25.41

(57) ABSTRACT

Disclosed herein are processes and devices for collecting nucleic acids of microorganisms from particulate samples.

### CROSS-REFERENCE TO RELATED APPLICATION

This application claims priority from U.S. Provisional Application Ser. No. 61/231,146 filed Aug. 4, 2009, which is incorporated by reference herein in its entirety.

### FIELD OF INVENTION

The field relates to a methods and devices for the isolation of nucleic acids from microorganisms contained in particulate samples.

### BACKGROUND OF INVENTION

The analysis of samples for microorganisms, such as bacteria, is important for public health. Foods grown, purchased, and consumed by the general population may contain or acquire microorganisms, which flourish or grow as a function of the environment in which they are located. This growth may lead to accelerated spoilage of the food product or the proliferation of pathogenic organisms, which may produce toxins or allergens.

One method of identifying microorganisms in particulate samples is through a molecular mechanism whereby the nucleic acids of such microorganism are detected. These detection methods occur with volumes of sample less than one milliliter whereas the target pathogen may exist in concentrations of one organism per 100 grams or more of sample. In order to increase the pathogen concentration to measurable levels, the particulate sample pathogens are enriched in a nutrient broth. Frequently the broth volumes range from 225 milliliters to four liters and the sample mass is 25 grams to 375 grams. A method is needed to concentrate the pathogens from this large, enriched volume to the detectable volume in order to shorten the enrichment time.

Concentration of the pathogen may be performed by capture of the whole pathogen organism through the use of affinity ligand-antigen interactions. For example, an antibody might be bonded to a magnetic particle (see, e.g., U.S. Pat. No. 7,507,528). After exposing this particle to the enriched sample, the target organism might be attached to the particle and then magnetically separated from the rest of the enrichment broth and thereby be isolated and concentrated. The shortcoming of this approach is that not all pathogens of a given strain such as the pathogenic forms of Escherichia coli express their pathogenicity in their antigens and therefore will not be collected. Examples are the rough strains of E. coli O157 (P. Feng et al., Identification of a Rough Strain of Escherichia coli O157:H7 That Produces No Detectable O157 Antigen, J. Clin. Microbiol. 1998 August; 36(8):2339-2341). Another potential shortcoming of the use of affinity ligands (particularly antibodies) arises due to the wide range of affinities of any given polyclonal or monoclonal antibody for target. Many antibodies targeting gram positive bacteria, for instance, suffer from low affinities resulting in decreased detection sensitivity. Decreased detection sensitivities translate to longer pre-enrichment time requirements, which can outweigh any potential gains achieved through sample concentration.

An alternative approach is to capture the free nucleic acids from a sample. Since the pathogenic nature of an organism is always contained in its DNA signature, this approach avoids the problems of poor or no expression on the surface of the organism of this property. Nucleic acid binding technologies are also usually highly efficient in overcoming the poor affinity issues associated with the use of antibodies. Because DNA detection is highly specific for the target structure, a non-specific binding technique may be used for this purpose.

A typical approach applied to the separation and isolation of nucleic acids from microorganisms is the use of nucleic acid binding materials. For example, one prominent example of nucleic acid binding material is a silica surface due to its ability to bind reversibly nucleic acids in the presence of chaotropic reagents (Vogelstein B. and Gillespie D., Proc. Natl. Acad. Sci. USA 76:615-19 (1979)). Such binding is assumed to be effected by oxidic surfaces (“X—OH”) interacting with phosphate groups of the nucleic acids. In physical structure, these silica surfaces have been varied. Non-magnetic silica particles have been used with centrifuges and non-particulate samples, but when food samples are present the food centrifuges with the particles, and a separation does not occur. Magnetic silica particles have been used but require the use of a magnetic system to concentrate the particles in a region of the container surface. Filtration of the sample through a silica filter is also possible, but in the case of a good sample the food particulates are also captured on the filter, and a separation does not occur.

### SUMMARY OF INVENTION

One aspect is for a process for collecting nucleic acids of microorganisms from a particulate sample comprising:
(a) transferring an aliquot of a particulate sample enrichment broth containing microorganisms into a device comprising
   (i) a chamber interior comprising a fibrous, non-magnetic nucleic acid binding surface, the chamber interior being capable of expanding in size in at least one dimension; and
   (ii) a nucleic acid binding solution;
   the fibers of the fibrous, non-magnetic nucleic acid binding surface expanding to at least partially fill the chamber interior upon wetting with the particulate sample enrichment broth and the nucleic acid binding solution;
(b) mixing the particulate sample enrichment broth containing microorganisms with the nucleic acid binding solution thereby lysing the microorganisms;
(c) expelling the nucleic acid binding solution from the device by compression of the fibrous, non-magnetic nucleic acid binding surface while retaining the fibrous, non-magnetic nucleic acid binding surface in the chamber interior;
(d) transferring an aliquot of elution buffer into the device;
(e) mixing the elution buffer with the fibrous, non-magnetic nucleic acid binding surface; and
(f) expelling the elution buffer from the device by compression of the fibrous, non-magnetic nucleic acid binding surface while retaining the fibrous, non-magnetic nucleic acid binding surface in the chamber interior, whereby the elution buffer contains nucleic acids from the microorganisms.

Another aspect is for a process for collecting nucleic acids of microorganisms from a particulate sample comprising:
(a) exposing an aliquot of a particulate sample enrichment broth containing microorganisms to a non-magnetic nucleic acid binding surface in the presence of a nucleic acid binding solution, said microorganisms comprising nucleic acids and said non-magnetic nucleic acid binding surface comprising a fibrous material capable of expansion upon wetting;
(b) lysing the microorganisms;
(c) binding the nucleic acids to the non-magnetic nucleic acid binding surface by expanding the fibrous material;
(d) separating the particulate sample enrichment broth from the non-magnetic nucleic acid binding surface comprising nucleic acids bound thereto by compression of the fibrous material;
(e) washing the non-magnetic nucleic acid binding surface with a wash solution whereby the fibrous material is again expanded;
(f) separating the wash solution from the non-magnetic nucleic acid binding surface comprising nucleic acids bound thereto by compression of the fibrous material;
(g) exposing an aliquot of elution buffer to the non-magnetic nucleic acid binding surface whereby the fibrous material is again expanded; and
(h) eluting nucleic acids from the non-magnetic nucleic acid binding surface with an elution buffer by compression of the fibrous material.

Other objects and advantages will become apparent to those skilled in the art upon reference to the detailed description that hereinafter follows.

### BRIEF DESCRIPTION OF THE FIGURES

FIG. 1A is a line graph showing PCR profiles of Salmonella samples (cell concentration of 10×10⁴ CFU/ml) mixed with 15% fat ground beef when collected by a syringe process described herein or by a standard BAX® procedure. FIG. 1B is a line graph showing PCR profiles of Salmonella samples (cell concentration of 10×10³ CFU/ml) mixed with 15% fat ground beef when collected by a syringe process described herein or by a standard BAX® procedure. FIG. 1C is a line graph showing PCR profiles of Salmonella samples (cell concentration of 10×10⁵ CFU/ml) mixed with 15% fat ground beef when collected by a syringe process described herein or by a standard BAX® procedure.

FIG. 2A is a line graph showing PCR profiles of Salmonella samples mixed with 15% fat ground beef when collected by a syringe process described herein, by a standard BAX® procedure, or by a spin preparation procedure. “C” spike level is the lowest cell level tested (1×10⁴ CFU/mL). FIG. 2B is a line graph showing PCR profiles of Salmonella samples mixed with 15% fat ground beef when collected by a syringe process described herein, by a standard BAX® procedure, or by a spin preparation procedure. “Unspiked” is the negative control.

### DETAILED DESCRIPTION OF PREFERRED EMBODIMENTS

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### Definitions

In this disclosure, a number of terms and abbreviations are used. The following definitions are provided.

As used herein, the term “about” or “approximately” means within 20%, preferably within 10%, and more preferably within 5% of a given value or range.

The term “comprising” is intended to include embodiments encompassed by the terms “consisting essentially of” and “consisting of”. Similarly, the term “consisting essentially of” is intended to include embodiments encompassed by the term “consisting of”.

“Polymerase chain reaction” is abbreviated PCR.

A “chaotrope” is any chemical substance which disturbs the ordered structure of liquid water. Chaotropes facilitate, e.g., unfolding, extension, dissociation of proteins, and the hydrogen boding of nucleic acids. Exemplary chaotropic salts include sodium iodide, sodium perchlorate, guanidinium thiocyanate, guanidinium isothiocyanate, and guanidinium hydrochloride.

The term “isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. The term also embraces recombinant polynucleotides and chemically synthesized polynucleotides.

The terms “polynucleotide”, “polynucleotide sequence”, “nucleic acid”, “nucleic acid sequence”, and “nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more strands of cDNA, genomic DNA, synthetic DNA, or mixtures thereof.

The term “silica” as used herein denotes materials which are mainly built up of silicon and oxygen. These materials comprise, for example, silica, silicon dioxide, silica gel, fumed silica gel, diatomaceous earth, celite, talc, quartz, crystalline quartz, amorphous quartz, glass, glass particles including all different shapes of these materials. Glass particles, for example, may comprise particles of crystalline silica, soda-lime glasses, borosilicate glasses, and fibrous, non-woven glass.

### Collection Methods

It was discovered that a particulate sample enrichment may be mixed with non-magnetic nucleic acid binding surface fibers in a chamber, and when that chamber is compressed and its contents released through an orifice, the fibers remain in the chamber whereas the particles are expelled. If this process is performed in the presence of a chaotrope, the non-magnetic nucleic acid binding surface fibers expand during mixing to infiltrate the bulk of the enrichment and collect a substantial portion of the nucleic acids. Also, after expulsion of the enrichment, and wash volumes may be introduced to the fiber with the non-magnetic nucleic acid binding surface fibers again expanding to the volume. When these wash volumes are expelled, remaining particulates are also expelled resulting in a small, compressed, mass of non-magnetic nucleic acid binding surface fibers being a very small volume of the original enrichment and presenting most of the target organism nucleic acids. The nucleic acids may be released from the non-magnetic nucleic acid binding surface fiber surface into a small volume of eluent producing a large concentration amplification of the target.

In one embodiment, an aliquot of a particulate sample enrichment broth containing microorganisms is transferred into a device comprising a chamber interior comprising a fibrous, non-magnetic nucleic acid binding surface, the chamber interior being capable of expanding in size in at least one dimension; and a nucleic acid binding solution; the fibers of the fibrous, non-magnetic nucleic acid binding surface expanding to at least partially fill the chamber interior upon wetting with the particulate sample enrichment broth and the nucleic acid binding solution. A typical sample enrichment protocol for diarrheagenic E. coli is described in the Bacteriological Analytical Manual, “BAM” (U.S. Food and Drug Administration): Aseptically weigh 25 g of sample into 225 ml of Brain Heart Infusion (BHI) broth (dilution factor of 1:10). If necessary, sample size may deviate from 25 g depending on availability of the sample, as long as the diluent is adjusted proportionally. Blend or stomach briefly. Incubate the homogenate for 10 min at room temperature with periodic shaking then allow the sample to settle by gravity for 10 min. Decant medium carefully into a sterile container and incubate for 3 h at 35° C. to resuscitate injured cells. Transfer contents to 225 mL double strength Tryptone Phosphate (TP) broth in a sterile container and incubate 20 h at 44.0±0.2° C. It is noted, however, that any sample enrichment procedure can be utilized in the present processes.

Inside the chamber resides a fibrous, non-magnetic nucleic acid-binding surface. In some embodiments this fibrous, non-magnetic nucleic acid-binding surface is a clean silica surface, with some embodiments utilizing a clean, activated silica surface. Cleaning and activation of the silica is effectuated, e.g., by washing with hydrocholoric acid although a separate cleaning step may not be required for all silica types. Following the cleaning step the cleaning solution is expelled and the enriched particulate sample, e.g. a food sample or a clinical sample, containing the target organism of interest is aspirated.

Alternatively, the non-magnetic nucleic acid-binding surface can be, e.g., NOMEX® fibers (meta-aramid; E.I. du Pont de Nemours & Co., Wilmington, Del.), KEVLAR® (para-aramid; E.I. du Pont de Nemours & Co., Wilmington, Del.), a polyamide, e.g., nylon (e.g., nylon 6,6, nylon 6, nylon 11, nylon 12, nylon 612).

Typically, the volume of sample may range from 500 μL to 10 mL or higher depending on sample type. An equal volume of nucleic acid binding solution containing, in some embodiments, detergent, ethylenediaminetetraacetic acid (EDTA), buffering components and possibly other components to facilitate binding and lysis is then aspirated with the sample. The nucleic acid binding solution is typically a chaotropic salt but alternatively can be a blend of salts such as 6 M NaClO₄, Tris, and trans-1,2-cyclohexanediaminetetraacetic acid (CDTA); or 8 M NaClO₄ and Tris at pH 7.5; or NaI.

Mixing the particulate sample enrichment broth containing microorganisms with the nucleic acid binding solution thereby lyses the microorganisms. The solutions within the chamber are mixed thoroughly (on, for example, a vortex mixer) causing the fibrous, non-magnetic nucleic acid-binding surface to disperse fully and expose the bulk of its surface area throughout the liquid in the chamber. The device is then incubated during which time cell lysis and nucleic acid binding occurs. An exemplary mixing condition is at room temperature for 15 minutes on a rotating mixer.

After mixing, the nucleic acid binding solution is expelled from the device by compression of the fibrous, non-magnetic nucleic acid binding surface while retaining the fibrous, non-magnetic nucleic acid binding surface in the chamber interior.

As an example, using the chamber described above, an aliquot of a food sample incubation broth containing microorganisms is pulled into the chamber of the device, and the broth is mixed with the nucleic acid binding solution within the chamber. After mixing, the nucleic acid binding solution is expelled from the chamber, but nucleic acids within the microorganisms bind to the fibrous, non-magnetic nucleic acid-binding surface and are thereby retained within the chamber.

In some embodiments, the fibrous, non-magnetic nucleic acid binding surface can then be washed with a wash solution. Following the initial sample incubation after the liquid is expelled from the device, an equal volume of wash buffer containing the nucleic acid binding solution, detergent, and buffering salts can be aspirated into the device. The device can then be mixed, e.g., on a vortex mixer as before, to expose fully the fibrous, non-magnetic nucleic acid binding surface, and the wash fluid is then immediately expelled, while retaining the fibrous, non-magnetic nucleic acid binding surface in the chamber interior.

Further, an equal volume of ethanol (e.g., 70% ethanol) can then be aspirated, mixed as before, and expelled. Next, an equal volume of acetone can be aspirated, mixed as before, and expelled.

The wash solution is selected such that a release of the nucleic acids from the fibrous, non-magnetic nucleic add binding surface preferably does not take place—or at least not in any significant amount—yet any impurities present are washed out as well as possible. The contaminated wash solution is preferably removed in the same manner as the nucleic acid binding solution at the end of the binding of the nucleic acids.

Any conventional wash buffer or any other suitable medium can be used as wash solution. Generally buffers with low or moderate ionic strength are preferred such as, for example, 10 mM Tris-HCl at a pH of 8, 0-10 mM NaCl. In addition, however, wash buffers that have higher salt concentrations—such as, for example, 3 M guanidinium hydrochloride—can also be used. Equally, other standard media for carrying out the washing step can be used, for example acetone or alcohol containing media such as, for example, solutions of lower alkanols with one to five carbon atoms, preferably solutions of ethanol in water and especially preferred aqueous 70% ethanol. In another preferred embodiment, the wash solution is isopropanol.

In some embodiments, the washing solution is characterized in that the nucleic acid binding solution, particularly a chaotropic substance, is not contained therein. As a result, a possibility of having the chaotropic substance incorporated into a recovery step after the washing step can be reduced. In the elution step, where the chaotropic substance is incorporated thereinto, the chaotropic substance sometimes hinders a later enzyme reaction such as PCR reaction or the like; therefore considering the later enzyme reaction, not including the chaotropic substance to a washing solution is preferable.

Following expulsion of the nucleic acid binding solution from the chamber, or following the optional wash step(s) if utilized, the fibrous, non-magnetic nucleic acid binding surface can be dried by placing the device in, e.g., a heat block (e.g., at 55° C. for 15 minutes). In another embodiment, the optional drying step can be performed by use of a vacuum.

Following the optional drying step, the captured nucleic acid is eluted by exposure to a small volume (e.g., 70-100 μl) of an elution buffer. A typical elution buffer is a PCR buffer solution pH 8.3 containing all the necessary components for PCR (i.e., MgCl₂, buffering salts, etc.) but not containing a chaotropic salt. The selection of the elution buffer is determined in part by the contemplated use of the isolated nucleic acids. Examples of suitable elution buffers are TE buffer, aqua bidest and PCR buffer. It is preferred that pH of a elution solution is 2 to 11 and, more preferably, 5 to 9. In addition, ionic strength and salt concentration particularly affect the elution of adsorbed nucleic acid. Preferably, the elution solution has an ionic strength of 290 mmol/L or less and has a salt concentration of 90 mmol/L or less. As a result thereof, recovering rate of nucleic acid increases and much more nucleic acid is able to be recovered.

When mixing is complete, the elution buffer is expelled from the syringe, with the elution buffer containing nucleic acids that previous were bound to the nucleic acid-binding surface. As necessary, additional elution buffer can be pulled into the syringe, mixed with the nucleic acid-binding surface, and expelled from the syringe to maximize recovery of bound nucleic acids. Because the solution resulting from the contact with the nucleic acid-binding surface contains the adsorbed nucleic acids, the recovered solution is typically subjected to a following step, for example PCR amplification of the nucleic acids.

Also, in the elution step, it is possible to add a stabilizing agent for preventing degradation of nucleic acid recovered in the elution solution of nucleic acid. As the stabilizing agent, an antibacterial agent, a fungicide, a nucleic acid degradation inhibitor and the like can be added. As the nuclease inhibitor, EDTA and the like can be cited.

There is no limitation for the infusing times for a elution buffer and that may be either once or plural times. Usually, when nucleic acid is to be separated and purified quickly and simply, that is carried out by means of one recovery while, when a large amount of nucleic acid is to be recovered, elution buffer may be infused for several times.

By way of the above methods, nucleic acids can be recovered from a myriad of microorganisms including, e.g., bacteria, fungi, algae, or viruses.

### EXAMPLES

The present invention is further defined in the following Examples. It should be understood that these Examples, while indicating preferred embodiments of the invention, are given by way of illustration only.

The meaning of abbreviations is as follows: “h” means hour(s), “min” means minute(s), “sec” means second(s), “d” means day(s), “mL” means milliliter(s), “μL” means microliter(s), “CFU” means colony forming unit(s), “MP” means multiplex, “BHI” means brain heart infusion, “BPW” means buffered peptone water, “n.d.” means no data.

### Example 1

### Standard BAX® Protocol vs. Silica Wool Chaotrope Capture Process in Ground Beef Post Enrichment Spiked with Salmonella

An overnight culture of Salmonella DD1261 was grown in BHI medium to 1.0×10⁹ CFU/mL, and then 40 μL of cells were spiked into 4 mL sample aliquots. Separately, 25 g of 15% fat ground beef was incubated in 225 mL BAX® MP medium (E.I. du Pont de Nemours & Co., Wilmington, Del.) at 37° C. overnight. The Salmonella DD1261 cells were then mixed briefly with the ground beef in MP medium and the sample was allowed for more than 4 min. 4 mL aliquots were removed from just below surface of enrichment.

From the mixture was taken two 5 μL aliquots that were mixed with 200 μL BAX® lysis buffer with ProE and incubated for 20 min at 37° C. then 10 min at 95° C. 50 μl of the BAX® lysis buffer/Salmonella/beef mixture was loaded onto BAX® Salmonella tablets and run on a BAX® System Q7.

Separately, 1 mL aliquot for a syringe sample preparation was taken from the Salmonella/beef mixture. The syringe was prepared by placing 10 mg of Quartzel® wool (fused quartz fiber, 4 μm; Saint-Gobain Quartz, Louisville, Ky.) in a 3 ml syringe. Approximately 1 ml Lysis buffer L6 and the approximately 1 ml of the syringe sample from the MP Enrichment was premixed and then aspirated into the syringe. The aspirate was vortex mixed and then incubated for 15 min on the mixer. The fluid in the syringe was then ejected completely from the syringe.

Next, approximately 2 ml wash buffer L2 was aspirated into the syringe, vortex mixed, and the fluid then ejected completely from the syringe.

Approximately 2 ml 70% EtOH was then aspirated into the syringe, vortex mixed, and the fluid then ejected completely from the syringe.

Next, approximately 2 ml acetone was aspirated into the syringe, vortex mixed, and the fluid ejected completely from the syringe. The syringe was then incubated in a heat block without the plunger at 55° C. for 15 min to dry the Quartzel® wool.

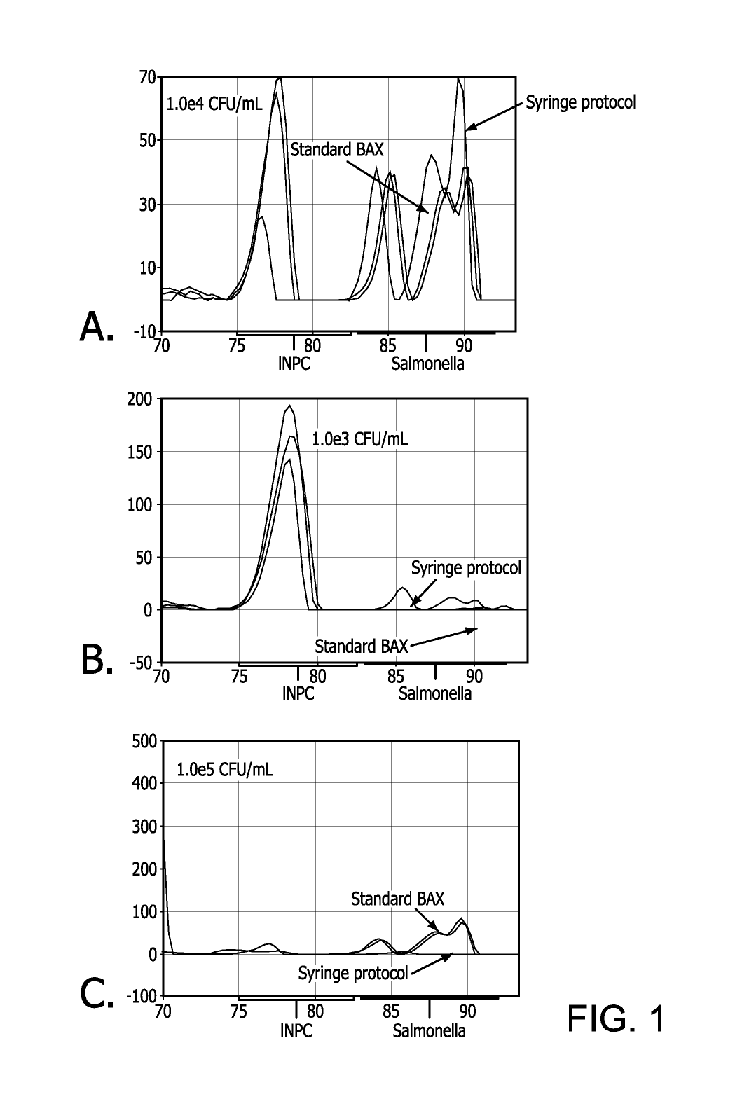

After PCR, the chaotrope capture syringe process resulted in a more sensitive detection than the standard BAX® protocol (See FIG. 1A-B). The chaotrope protocol was able to detect the Salmonella at 1.0×10³ CFU/ml while the standard BAX® protocol was not. In addition the chaotrope protocol gave a larger target peak at the 1.0×10⁴ CFU/ml level compared to the standard BAX® protocol.

Note that it is unclear why, at the 1.0×10⁶ CFU/ml level, the syringe protocol did not generate any target or internal positive control (INPC) peak (FIG. 1C). The reaction appeared to be inhibited.

### Example 2

### Centrifugation Sample Prep Evaluation in Ground Beef Post Enrichment Spike with Salmonella. Evaluation with Lyospheres Supplemented with Tag vs. Silica Wool Chaotropic Salt Syringe Protocol

An overnight culture of Salmonella DD1261 was grown in BHI medium to 1.0×10⁹ CFU/mL, and then 40 μL of cells were spiked into 4 mL sample aliquots. Separately, 25 g of 15% fat ground beef was incubated in 225 mL BPW medium at 37° C. overnight. The Salmonella DD1261 cells were then mixed briefly with the ground beef in BPW medium and the sample was allowed to settle for more than 4 min. 4 mL aliquots were removed from just below surface of enrichment.

From the mixture was taken a 5 μL aliquot that was mixed with 200 μL BAX® lysis buffer with ProE and incubated for 20 min at 37° C. then 10 min at 95° C. 30 μl of the mixture was then loaded onto lyospheres DPQ-TC and 0.75 μL Amplitaq was added thereto.

Two separate 300 μL aliquots of the Salmonella/beef mixture were loaded into 1.5 ml microfuge tube containing 500 μL of sample preparation reagent (8 ml BAX® lysis buffer, 80 μL of 100% Triton X100, 40 μL 20% SDS solution). The microfuge tube was then spun 3 min at 12,000 g, the supernatant removed completely by aspiration, the pellet resuspended in 200 μL BAX® buffer with protease, and incubated for 20 min at 37° C. then 10 min at 95° C. 30 μl of the mixture was then loaded onto lyospheres DPQ-TC and 0.75 μL Amplitaq was added thereto.

Separately, 1 mL aliquot for a syringe sample preparation was taken from the Salmonella/beef mixture. The syringe was prepared by placing 10 mg of Quartzel® wool (fused quartz fiber, 4 μm; Saint-Gobain Quartz, Louisville, Ky.) in a 3 ml syringe. Approximately 1 ml Lysis buffer L6 and the approximately 1 ml of the syringe sample from the BPW Enrichment was premixed and then aspirated into the syringe. The aspirate was vortex mixed and then incubated for 15 min on the mixer. The fluid in the syringe was then ejected completely from the syringe.

Next, approximately 2 ml wash buffer L2 was aspirated into the syringe, vortex mixed, and the fluid then ejected completely from the syringe.

Approximately 2 ml 70% EtOH was then aspirated into the syringe, vortex mixed, and the fluid then ejected completely from the syringe.

Next, approximately 2 ml acetone was aspirated into the syringe, vortex mixed, and the fluid ejected completely from the syringe. The syringe was then incubated in a heat block without the plunger at 55° C. for 15 min to dry the Quartzel® wool.

After drying, 100 μl of BAX® buffer (preheated to 55° C.) was aspirated into the syringe, the plunger reinserted, and as much liquid as possible was ejected from the syringe. 30 μl of the ejected liquid was then loaded onto lyospheres DPQ-TC and 0.75 μL Amplitaq was added thereto.

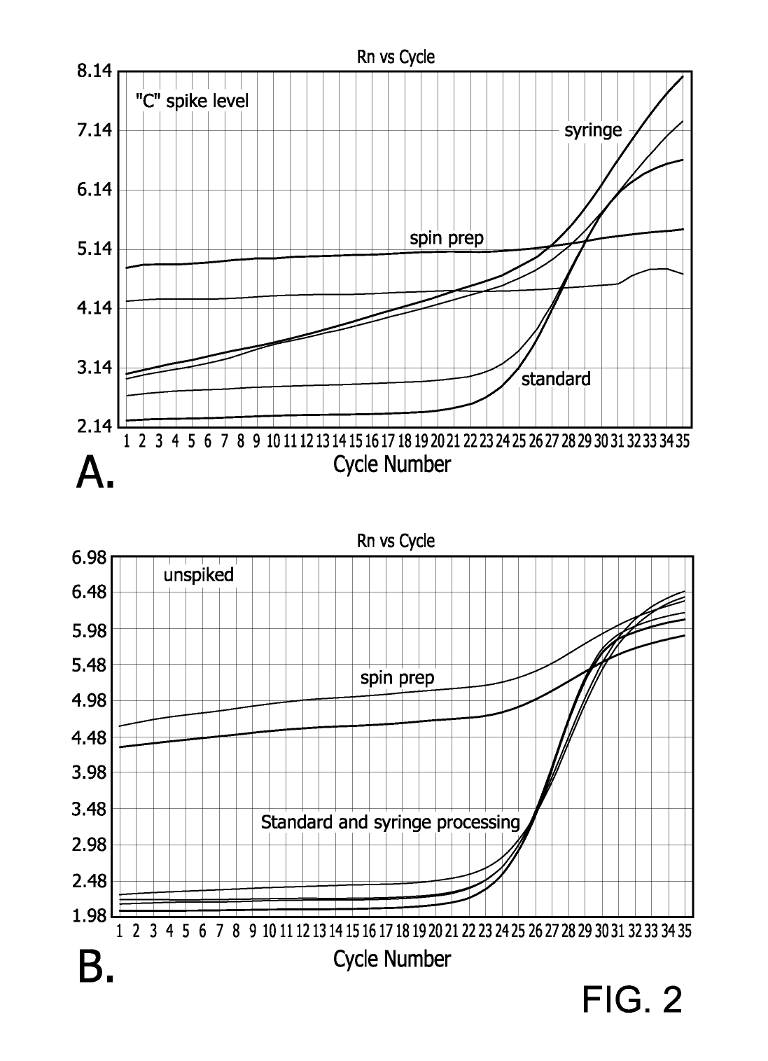

INPC response indicated that overall the spin preparation resulted in higher levels of inhibition than the other two methods. The “C” spike level showed inhibition for both the spin prep and syringe methods (see FIG. 2A). Visual inspection revealed that the “C” spike tube was much more turbid than the other tubes and the pellet was much larger for the spin preparation method (which showed the most inhibition). This may have impacted the results at the “C” level.

Unspiked data demonstrated that the beef was not naturally contaminated with Salmonella.

As noted in Table 1, the spin preparation method was 1-2 logs more sensitive than the standard BAX® method based on Ct (Crossing threshold; a measure measure of PCR efficiency and starting target copy number. The lower the Ct the more target was in the reaction to start with and/or the more efficient was the reaction. 3Ct=1 log). The syringe method was 3-4 logs more sensitive than the standard BAX® method based on Ct.

TABLE 1

| CFU/ml | Treatment | Avg. Ct. (n = 2) |
|---|---|---|
| 1.00 × 10⁶ | Standard (5 μl) | 29 |
| | Spin Prep (300 μl) | 24.3 |
| | Syringe (1 ml) | 20.4 |
| 1.0 × 10⁵ | Standard (5 μl) | 34 |
| | Spin Prep (300 μl) | 28.1 |
| | Syringe (1 ml) | 22.5 |
| 1.0 × 10⁴ | Standard (5 μl) | n.d. |
| | Spin Prep (300 μl) | n.d. |
| | Syringe (1 ml) | n.d. |

Interestingly the syringe method performed well even though Applicants neglected to add supplemental MgCl₂ to the L2 buffer. Thus, when food matrix is present, the additional Mg supplement may be unnecessary. Also, given that the DNA release from the silica is known to be suboptimal even better results may be possible in the future.

What is claimed is:

1. A process for collecting nucleic acids of microorganisms from a particulate sample comprising:
(a) transferring an aliquot of a particulate sample enrichment broth containing microorganisms into a device comprising
(i) a chamber interior comprising a fibrous, non-magnetic nucleic acid binding surface, the chamber interior being capable of expanding in size in at least one dimension; and
(ii) a nucleic acid binding solution;
the fibers of the fibrous, non-magnetic nucleic acid binding surface expanding to at least partially fill the chamber interior upon wetting with the particulate sample enrichment broth and the nucleic acid binding solution;
(b) mixing the particulate sample enrichment broth containing microorganisms with the nucleic acid binding solution thereby lysing the microorganisms;
(c) expelling the nucleic acid binding solution from the device by compression of the fibrous, non-magnetic nucleic acid binding surface while retaining the fibrous, non-magnetic nucleic acid binding surface in the chamber interior;
(d) transferring an aliquot of elution buffer into the device;
(e) mixing the elution buffer with the fibrous, non-magnetic nucleic acid binding surface; and
(f) expelling the elution buffer from the device by compression of the fibrous, non-magnetic nucleic acid binding surface while retaining the fibrous, non-magnetic nucleic acid binding surface in the chamber interior, whereby the elution buffer contains nucleic acids from the microorganisms.

2. The process of claim 1 comprising after step (c) and before step (d) the further steps of:
washing the fibrous, non-magnetic nucleic acid binding surface with a wash solution; and
expelling the wash solution by compression of the fibrous, non-magnetic nucleic acid binding surface while retaining the fibrous, non-magnetic nucleic acid binding surface in the chamber interior.

3. The process of claim 2, wherein the expelling is performed by vacuum.

4. The process of claim 1, wherein the expelling of step (c), (f), or (c) and (f) is performed by vacuum.

5. The process of any of claims 1-4, wherein the fibrous, non-magnetic nucleic acid binding surface is a clean silica surface.

6. The process of claim 5, wherein the clean silica surface is a clean, activated silica surface.

7. The process of claim 6, wherein the clean, activated silica surface comprises silica wool.

8. The process of any of claims 1-4, wherein the fibrous, non-magnetic nucleic acid binding surface is selected from the group consisting of a meta-aramid surface, a para-aramid surface, and a polyamide surface.

9. The process of any of claims 1-4, wherein the nucleic acid binding solution comprises a chaotropic salt.

10. The process of any of claims 1-4, wherein the microorganisms are bacteria, fungi, algae, or viruses.

11. The process of any of claims 1-4, wherein the particulate sample is a food sample or a clinical sample.

12. A process for collecting nucleic acids of microorganisms from a particulate sample comprising:
(a) exposing an aliquot of a particulate sample enrichment broth containing microorganisms to a non-magnetic nucleic acid binding surface in the presence of a nucleic acid binding solution, said microorganisms comprising nucleic acids and said non-magnetic nucleic acid binding surface comprising a fibrous material capable of expansion upon wetting;
(b) lysing the microorganisms;
(c) binding the nucleic acids to the non-magnetic nucleic acid binding surface by expanding the fibrous material;
(d) separating the particulate sample enrichment broth from the non-magnetic nucleic acid binding surface comprising nucleic acids bound thereto by compression of the fibrous material;
(e) washing the non-magnetic nucleic acid binding surface with a wash solution whereby the fibrous material is again expanded;
(f) separating the wash solution from the non-magnetic nucleic acid binding surface comprising nucleic acids bound thereto by compression of the fibrous material;
(g) exposing an aliquot of elution buffer to the non-magnetic nucleic acid binding surface whereby the fibrous material is again expanded; and
(h) eluting nucleic acids from the non-magnetic nucleic acid binding surface with an elution buffer by compression of the fibrous material.
